(19) United States
(12) Patent Application Publication
(10) Pub. No.: US 2003/0166027 A1
(43) Pub. Date: Sep. 4, 2003

(54) Compositions, test kits and methods for detecting helicobacter pylori

(76) Inventors: George Sachs, Encino, CA (US); Petra Voland, Munich (DE)

Correspondence Address: Gabor L. Szekeres, 8141 E. KAISER BOULEVARD, SUITE 112, ANAHEIM, CA, 92808, US

(21) Appl. No.: 10/080,113

(22) Filed: Feb. 21, 2002

(51) Int. Cl. G01N 33/554; G01N 33/569; C07K 14/195

(52) U.S. Cl. 435/7.32; 530/350

(57) ABSTRACT

Four proteins are obtained from H. pylori bacteria each of which has regions which act as antigens specific to H. pylori. The proteins are isolated, purified and designated HP1, HP2, HP3, and HP4 with respective molecular weights of 32 kd, 30 kd, 23 kd and 15 kd. An assay, a method and a kit is developed utilizing a combination of at least three of these protein to detect the presence of antibodies to H. pylori in human sera. The method of detection is quantified and suitable for monitoring the eradication of H. pylori bacteria by drug therapy of human patients infected by these bacteria.

### BACKGROUND OF THE INVENTION

1. Field of the Invention

The invention relates to an antigen composition that can detect the presence of antibodies specific to Helicobacter pylori. The invention also relates to a method for the preparation of the antigens and the composition and a method and kit for detecting the presence of the Helicobacter pylori-specific antibodies. The method also is able to detect eradication of the organism, providing novel methodology.

2. Brief Description of Background Art

Helicobacter pylori (formerly Campylobacter pylori), hereinafter also referred to as H. pylori, was discovered by B. J. Marshall et al. in 1983. It is a gram-negative, spiral shaped, motile bacterium that colonizes the human stomach that more than 50% of the world's adult population in industrial countries and almost 100% in developing countries are infected with. In association with the infection, gastric disorders like chronic gastritis, gastric and duodenal ulcer disease as well as gastric carcinoma occur.

The diagnosis of an infection with H. pylori is usually achieved in two ways. Directly (invasive) by endoscopic examination with biopsy, followed by histology and culture of the bacterium and indirectly (non invasive) by testing peripheral blood or serum samples for antibodies against H. pylori or performing a ¹³C urea breath test (¹³C UBT).

Serological tests and the ¹³C UBT are the two non-invasive techniques, used in the management of H. pylori infection and eradication. The accuracy of a serological test is dependent on the nature of the antigen(s). Most of the serological tests are ELISA based and use whole cell lysates of H. pylori as the antigen, often in combination with a more purified antigen preparation like recombinant vacA, cagA and/or iceA protein. Using a crude lysate preparation of the whole organism can cause problems with the specificity of the test via nonspecific binding of antibodies not specific for H. pylori to components of the antigen preparation that might be present in other H. pylori related organisms (false positives). On the other hand crude antigen preparation might cause false negative results because unwanted components in the preparation might dilute specific antigens or interfere with the presentation of those required to determine infection. The use of a total protein isolate also prevents serology from detecting loss of the organism and therefore is not suitable for evaluating success of eradication therapy. The UBT gives false negatives when patients are taking proton pump inhibitor drugs (PPI's) due to inhibition of urease activity by neutral pH.

Currently, the role of serology in managing H. pylori infection is as a screening procedure and for diagnosis of infection but not for determination the success of eradication. That is because the tests are not designed to detect reductions in the antibody titer during the post eradication period. In contrast the ¹³C UBT is highly sensitive and specific but expensive and not available to all general physicians and is not an office procedure. There is an unsatisfied need for an easy non-invasive and sensitive test to both diagnose the infection and to determine eradication of H. pylori infection after treatment available as an office procedure to gastroenterologists.

The accuracy of IgG serology, and therefore the usefulness of that approach in monitoring therapy and to confirm H. pylori eradication has already been pointed out and shown by other authors. See the publications by: Hirschl et al., The J. of Infect. Diseases, 1993, 168: 763-766; Lerang et al., Scand. J. Gastroenterol., 33(7):710-715, 1998; Cullen et al., The Lancet, Nov. 7, 1992, 340:1162-1163; Kosunen et al., The Lancet, April 11,339: 1992, 893-895. In a recent report an immunodominant outer membrane protein of H. pylori has been successfully used to assess the early response to eradication therapy in patients on a serological basis. See the publication by Nishizono. et al., Clin. and Diagn. Lab. Immunology, 1998, 5: 56-861.

The identification of unique H. pylori proteins/antigens others than cagA, vacA and iceA that can be used for diagnosis of H. pylori infection and for monitoring the success of eradication therapy in patients using a Western blot based method is therefore highly desirable.

U.S. Pat. No. 5,846,751 is related to a sensitive and specific antigen preparation for the detection of H. pylori in biological samples. The preparation uses a range of antigens derived from size exclusion chromatography of detergent-solubilized H. pylori cells. U.S. Pat. No. 5,459,041 discloses an antigenic composition for detecting the presence of antibodies specific for H. pylori wherein said antigen is a surface structure resolving into bands migrating at 63,000; 57,000 and 31,000 dalton bands when electrophoresed on sodium dodecyl sulfate polyacrylamide gel. U.S. Pat. No. 5,859,219 relates to a purified vacuolating toxin from H. pylori and methods to use same.

A PCT International Publication WO 00/56769 purports to describe an assay and method to satisfy the need for accurate diagnosis of H. pylori infection and for monitoring the success of eradication therapy in patients. However, the antigen proteins described in this publication are not in fact suitable for use in the assay, and therefore the assay and methods of this publication fail to accomplish their intended purpose.

### SUMMARY OF THE INVENTION

The subject invention has several distinct aspects. One aspect is a composition of antigens from H. pylori present in the lysate of whole bacterial cell preparations that is capable of detecting the presence or absence of specific antibodies against H. pylori with high accuracy and reliability. Another aspect is a method for the preparation of such a composition. A further aspect is a method for detecting the presence of antibodies resulting from Helicobacter pylori infection in a biological sample which makes use of such a composition. In particular the method relates to monitoring the success of eradication treatment of Helicobacter pylori. An additional aspect of the invention is a kit for determining the presence of antibodies formed in response to Helicobacter infection in a biological sample, the kit comprising such a composition.

### BRIEF DESCRIPTION OF THE DRAWINGS

FIG. 1A is titled “Reactivities of H. pylori positive sera with antigens from Hp504” and 1B is titled “Reactivities of H. pylori positive sera with antigens from Hp504.” These figures show the average titers of specific antibodies, expressed as percent Integrated Optical density (IOD), with HP1, HP2, HP3 and HP4 from H. pylori strain Hp504 (ATCC#43504) present in sera from 9 patients diagnosed with a H. pylori infection achieved in two independent experiments. The serum samples from each patient were obtained before, 3 months and 5 months after eradication therapy.

FIG. 2 is titled “Reactivities of H. pylori positive sera with antigens from Hp08.” It shows the average titers of specific antibodies, expressed as percent IOD, with HP1, HP2, HP3 and HP4 from another H. pylori strain, Hp08 (clinical isolate) present in sera from 9 patients diagnosed with H. pylori infection. The serum samples from each patient were obtained before, 3 months and 5 months after eradication therapy.

FIG. 3 is titled “Reactivities of H. pylori positive sera with antigens from Hp02.” It shows the average titers of specific antibodies, expressed as percent IOD, with HP1, HP2, HP3 and HP4 from still another H. pylori strain Hp02 (clinical isolate) present in sera from 9 patients diagnosed with H. pylori infection. The serum samples from each patient were obtained before, 3 months and 5 months after eradication therapy.

FIG. 4 is titled “Reactivities of H. pylori positive sera with antigens from Hp504, Hp08 and Hp02.” It summarizes the data shown in the previous figures. It shows the average titers of specific antibodies, expressed as percent IOD, with HP1, HP2, HP3 and HP4 from all three different H. pylori strains Hp504, Hp08 and Hp02 present in sera from 9 patients diagnosed with a H. pylori infection. The serum samples of each patient were obtained before, 3 months and 5 months after eradication therapy.

### DETAILED DESCRIPTION

The composition according to the invention comprises at least three Helicobacter pylori derived proteins or their antigenic regions, wherein the proteins are selected from the group of Helicobacter pylori derived proteins which are identified by SDS PAGE to consist of antigens specific to Helicobacter pylori of molecular weights 32 kd; 30 kd; 23 kd; and 15 kd. These antigens from H. pylori have not been used in this combination in other available tests. These proteins were assigned the following names:

HP1 32 kd protein;

HP2 30 kd protein;

HP3 23 kd protein, and

HP4 15 kd protein.

The antigens were identified by 2D gel electrophoresis and mass spectrometry and expression of recombinant proteins followed by Western analysis. The genomic data base (see Tomb et al., Nature, 388, 539-547 1997) identified HP1 as HpaA-neuraminyl-lactose-hemagglutinin precursor, HP2 as Omp18-peptido-glycan associated lipoprotein precursor, HP3 as HP0596 a hypothetical protein and HP4 RPL7/L12 50S ribosomal protein L7/L12.

The amino acid sequences of these proteins, based on the genomic data base, are as follows:

HP1 (HpaA-neuraminyl-lactose-hemagglutinin precursor):

1 mkkgslaivl gsllasgafy taladgmpak qqhnntgesv elhfhypikg kqepknshlv

61 vliepkiein kvipesyqke fekslflqls sflerkgysv sqfkdaseip qdikekallv

121 lrmdgnvail ediveesdal seekvidmss gylnlnfvep ksediihsfg idvskikavi

181 ervelrttns ggfvpktfvh riketdhdqa irkimnqayh kvmvhitkel skkhmehyek

241 vssemkkrk

HP2 (Omp18-peptido-glycan associated lipoprotein precursor):

1 mkrssvfsfl vafllvagcs hkmdnktvag dvsaktvqta pvttepapek eepkqepapv

61 veekpavesg tiiasiyfdf dkyeikesdq etldeivqka kenhmqvlle gntdefgsse

121 ynqalgvkrt lsvknalvik gvekdmikti sfgetkpkca qktrecyken rrvdvklmk

HP3 (HP0596)

1 mleksflksk qlflcglgvl mlqactcpnt sqrnsflqdv pywmlqnrse yitqgvdssh

61 ivdgkkteei ekiatkrati rvaqnivhkl keaylsktnr ikqkitnemf iqmtqpiyds

121 lmnvdrlgiy inpnneevfa lvrargfdkd alseglhkms ldnqavsilv akveeifkds

181 vnygdvkvpi am

HP4 (RPL7/L12 50S ribosomal protein L7/L12)

1 mnisvnpylm avvfvvfvll lwamnvwvyr pllafmdnrq aeikdslaki ktdnaqsvei

61 ghqieallke aaekrreiia eaiqkatesy davikqkene lnqefeafak qlqnekqalk

121 eqlqaqmpvf edelnkrvam glgs

In a preferred embodiment of the invention at least three of the four antigens HP1, HP2, HP3 and HP4 are present in the composition. In another preferred embodiment all four of these antigens are present. The 3 or 4 antigens can be present in the composition as a mixture, but preferably they are present as a combination.

Preferably the antigens in the composition according to the invention are present attached to a solid phase. In connection with the invention a solid phase preferably relates to a solid phase suitable for attachment of antigens, such as microtiter plates or membranes such as nitrocellulose and PVDF membranes. The antigens can be attached to the solid phase as a mixture. However, preferably they are attached in distinct locations (for example as separate spots or strips attached to a plate or membrane), thus forming a combination and not a mixture. A person having ordinary skill in the art will know on the basis of this description how to attach the three or four antigenic proteins of the invention to a solid phase such as a nitrocellulose and PVDF membrane.

In a preferred embodiment of the invention the composition according to the invention can be obtained by preparing a lysate of whole bacterial cell preparations of Helicobacter pylori and subjecting the lysate to gel separation. After separation the antigens may be transferred onto a solid phase, for example by electrotransfer to membranes. In another embodiment of the invention the proteins of the composition can be prepared according to recombinant methods. This can be achieved by cloning the complete sequence coding for the antigen(s) or part of it into an appropriate expression vector for an Escherichia coli expression system. These systems depend on expression of the protein of interest by induction of a system integrated promoter. After expression of the protein in high amounts it can be isolated and purified by affinity chromatography because it was expressed as a fusion protein or because flag has been attached to it. The possibilities of isolation and purification are entirely depending on the chosen system. In the case that only parts of the sequence of a protein are used for recombinant expression, an antigenicity plot has to be performed to make sure that highly antigenic regions of the protein are not lost thereby losing the capability of immuno reactivity with the specimen to be tested. A person having ordinary skill in the art will know on the basis of this description how to isolate the antigenic proteins of the invention from Escherichia coli expression system, and how to perform the antigenecity plot.

A further aspect of the invention relates to a method for detecting the presence of antibodies resulting from Helicobacter pylori infection in a biological sample. The method comprises the steps of:

(a) contacting the sample with a composition according to the invention;

(b) permitting the sample and said composition to form an antigen-antibody complex with respect to any antibody specific for said antigens of the composition contained in the sample;

(c) detecting the presence of any formed antigen-antibody complex denoting the presence of Helicobacter pylori infection.

Preferably in the method of the invention as well, the composition contains three (3) of the four (4) proteins, not as a mixture, but as a combination so that the formation (or lack thereof) of each antigen-antibody complex is detected separately.

A biological sample in connection with the invention is preferably human sera, because a principal application of the invention is to diagnose H. pylori infection in humans, and/or to monitor the eradication of H. pylori from human patients by drug therapy.

For detection of the presence of any formed antigen-antibody complex in step (c) it is preferred to use gold label or enzyme conjugated antibody, in particular an anti-Human IgG antibody. The person skilled in the art is familiar what kind of gold label or enzyme conjugated anti-Human IgG antibody can be used in connection with the detection of a said antigen-antibody complex. Anti-Human IgG antibodies which are conjugated to horseradish peroxidase are mentioned by way of example and as a preferred embodiment.

A further embodiment of the invention is a kit for determining the presence of antibodies formed in response to Helicobacter pylori infection in a biological sample, the kit comprising a composition according to the invention preferably attached to a solid support. Optionally the kit may comprise additional components such as a positive control (human serum containing antibodies against H. pylori), buffer solutions, suitable gold label antibody or an enzyme conjugated anti-Human IgG antibody and a suitable enzyme substrate. In a preferred embodiment of the invention the kit comprises a test strip wherein a composition according to the invention is attached to a nitrocellulose membrane and a suitable gold label antibody is used for detection of the presence of any formed antigen-antibody complex. Again, preferably the composition (itself comprising at least three of the four antigenic proteins) is attached to the membrane as a combination and not as a mixture, that is to say each antigenic protein is attached to the membrane at a separate location. After contacting the test strip with the biological sample the formation of a coloured line will denote the presence of Helicobacter pylori infection. A person skilled in the art is familiar with such type of test strip. This format of test strip is, for example, widely used in pregnancy hCG tests.

The method for detecting the presence of antibodies resulting from Helicobacter pylori infection is particularly suitable for determination of the eradication of Helicobacter pylori during and after eradication treatment as it allows to detect reductions in the antibody titer during the post eradication period. This method comprises the steps of:

(a) diagnosis of infection with H. pylori;

(b) monitoring antibody titers during eradication treatment;

(c) determination the eradication of the infection after eradication therapy,

wherein at least in steps (b) and (c) the presence or absence of antibodies resulting from H. pylori infection is determined by a method according to the invention.

Further objects and aspects of the invention will be evident from the ensuing description and claims.

### Materials and Methods

Materials: All materials used were of highest purity grade available.

Bacterial strains:

H. pylori strain ATCC#43504 (Hp504)(American Type Culture Collection, Rockville, Md.) and two clinical isolates, Hp08 and Hp02, were used as the source of H. pylori proteins. As a control for the specificity of the serological reactivities Campylobacter jejuni strain ATCC#29428 was included into the experiments.

Bacteria were grown on blood agar plates (BBL TSA 5% sheep blood, Becton Dickinson, Cockeysville, Md.) for 24 hr or in brain heart infusion (BHI) supplemented with 0.25% yeast extract (Difco Laboratories, Detroit, Mich.) and 6% horse serum (Gibco BRL, Grand Island, N.Y.) until reaching an OD₆₀₀ of 0.8-1.0 at 37° C. in a microaerobic atmosphere (5% O₂, 10% CO₂, 85% N₂. Bacteria grown in broth culture were collected by spinning for 10 min at 5000×g, washed once with phosphate buffered saline (PBS) pH 7.5 and then suspended in 1 ml ice cold deionized H₂O. Cells grown on up to three blood agar plates were harvested directly into 1 ml ice cold deionized H₂O. Lysis of the cells was obtained by three cycles in a French pressure cell with 20,000 psi at 4° C. The lysates were always kept on ice or at −20° C.

Human Sera and Antibodies Specific for H. pylori Proteins:

Human sera were provided by Dr. D. Vaira (S. Orsola Hospital, Bologna, Italy). We tested sera from nine with H. pylori infected patients (mean age 62.2, 5 female, 4 male; table 1) obtained before, 3 and 5 months after eradication therapy and sera from ten non-infected patients, (mean age 42.6, 5 female, 5 male; table 2) obtained before therapy. The H. pylori infection and the status of the gastrointestinal damage of all these individuals had been confirmed and examined by several assays (endoscopy, Clo, Colt, Histology, ELISA ¹³C UBT; table 1 and 2). The ELISA used for this purpose is described in Literature (Vaira et al., 1988a, 1988b, 1989, 1991, 1994a, 1994b; Oderda et al., 1989a, 1989b, 1991, 1992; Menegatti et al., 1995, 1996, 1998). According to these tests H. pylori infection has been eradicated after treatment in all patients.

As controls well characterized and specific polyclonal antibodies against a synthetic peptide of the urease B subunit from H. pylori (∝UreB#744, Byk Gulden, Konstanz, Germany), the urease A subunit from H. pylori (∝UreA#30588, Dr. H. Mobley, Univ. of Maryland, Baltimore, Md.) and a commercially available antiserum against the Hsp60 from Synechococcus sp. strain PCC 7942, (StressGen Biotechnologies Corp., Victoria, BC. Canada) (∝Hsp) were used. The latter detects specifically HspB of H. pylori.

The H. pylori eradication therapy comprises the administration of appropriate drugs to a patient in need of such treatment. This therapy per se is old in the art. As is known, the therapy may be conducted by administering to a patient amoxicilin in a dose of 1.0 g twice a day and omeprazole 20 mg once a day, for a total of seven days. It was such a therapy, with the above described doses and for the above-described duration which was monitored with the composition and method in accordance with the present invention. The therapy itself is shown in a summarized tabulated from below.

Table 1 titled “list of H. pylori infected patients discloses ages and gender, diagnostic test data and observation of medical conditions of 9 patients who were infected with H. pylori and who received the 7 day treatment described above. The patients were followed for five (5) months after termination of the therapy. ED at the heading of the fifth column of the table (and elsewhere) means erosive duodenitis. “Clo A”, “Clo C”, “Colt C” in column headings of Table 1 refer to tests well known in the art for the presence of H. pylori bacteria in samples taken by biopsy from the stomach of patients. “Histology A” in a column heading refers to histological examination of the biopsy samples from the stomach of the patients.

H. pylori Eradication Therapy:

| Substance | Dose |
|---|---|
| Amoxicillin | 1 g 2 × day |
| Clarithromycin: | 500 mg 1 × day |
| Omeprazole: | 20 mg 1 × day |
| Total duration | 7 days |
| of treatment: | |

“Giemsa C” in a column heading refers to a staining procedure designed to reveal the presence of bacteria. “IgG in a column heading refers to the quantitative immune response (expressed in IOD) obtained in accordance with the present invention. ¹³C UBT in a column heading refers to the prior art test described in the introductory section of the present application for patent. All of the tests and procedures, except the immunological test of the present invention, are well known in the state-of-the-art.

Table 2 is titled “list of non-infected patients”. This table discloses test results of 10 individuals who were not infected by H. pylori. It can be seen from these two tables that the results of the tests in accordance with the present invention (IgG column) are strongly positive in infected patients before therapy, are substantially negative in non-infected patients, and further that eradication of the bacteria by therapy results in significantly lower quantitative values in the IgG test of the invention, so that the course of the eradication of the bacteria can be monitored by the assay and method of the present invention.

TABLE 1
List of H. pylori infected patients

| | | | | | | Clo | Clo | Colt | Colt | Histology | | | | | |
|---|---|---|---|---|---|---|---|---|---|---|---|---|---|---|---|
| [t]therapy | Patient | Sex | Age | ED | | A | C | A | C | A | Glemsa A | Histology C | Glemsa C | IgG | ¹³C UBT |
| before | A3 808 | m | 45 | 1^{st} | AG | pos | pos | pos | pos | MIAG atr | 1 | MAG atr | 1 | 0.94 pos | 9.0 pos |
| | | | | 2^{nd} | N | neg | neg | neg | neg | N | 0 | N | 0 | n.d. | 0.0 neg |
| 3 months | | | | | n.d. | n.d. | n.d. | n.d. | n.d. | n.d. | n.d. | n.d. | n.d. | 0.88 pos | n.d. |
| 5 months | | | | | n.d. | n.d. | n.d. | n.d. | n.d. | n.d. | n.d. | n.d. | n.d. | 0.70 pos | n.d. |
| before | A3 828 | m | 69 | 1^{st} | N | pos | pos | pos | pos | MIAG | 1 | MIAG atr | 1 | 1.02 pos | 28.3 pos |
| | | | | 2^{nd} | AG | neg | neg | neg | neg | MIAG atr | 0 | MIAG | 0 | n.d. | 1.70 neg |
| 3 months | | | | | n.d. | n.d. | n.d. | n.d. | n.d. | n.d. | n.d. | n.d. | n.d. | 0.91 pos | n.d. |
| 5 months | | | | | n.d. | n.d. | n.d. | n.d. | n.d. | n.d. | n.d. | n.d. | n.d. | 0.87 pos | n.d. |
| before | A3 837 | f | 49 | 1^{st} | AG | pos | pos | pos | pos | MIAG | 1 | MIAG | 1 | 0.79 pos | 15.1 pos |
| | | | | 2^{nd} | N | neg | neg | neg | neg | N | 0 | MIAG | 0 | n.d. | 1.2 neg |
| | | | | | | | | | | | | atr IM | | | |
| 3 months | | | | | n.d. | n.d. | n.d. | n.d. | n.d. | n.d. | n.d. | n.d. | n.d. | 0.65 pos | n.d. |
| 5 months | | | | | n.d. | n.d. | n.d. | n.d. | n.d. | n.d. | n.d. | n.d. | n.d. | 0.24 neg | n.d. |
| before | A3 838 | f | 78 | 1^{st} | DE | pos | pos | pos | pos | MAG atr | 1 | N | 1 | 0.96 pos | 22.9 pos |
| | | | | 2^{nd} | DE | neg | neg | neg | neg | MIAG atr | 0 | N | 0 | n.d. | 1.5 neg |
| 3 months | | | | | n.d. | n.d. | n.d. | n.d. | n.d. | n.d. | n.d. | n.d. | n.d. | 0.89 pos | n.d. |
| 5 months | | | | | n.d. | n.d. | n.d. | n.d. | n.d. | n.d. | n.d. | n.d. | n.d. | 0.56 pos | n.d. |
| before | A4 012 | f | 57 | 1^{st} | DE | pos | pos | pos | pos | MAG atr | 1 | MAG atr | 2 | 0.83 pos | 48.2 pos |
| | | | | 2^{nd} | D | neg | neg | neg | neg | MIAG | 0 | MIAG atr | 0 | n.d. | 0.0 neg |
| 3 months | | | | | n.d. | n.d. | n.d. | n.d. | n.d. | n.d. | n.d. | n.d. | n.d. | 0.76 pos | n.d. |
| 5 months | | | | | n.d. | n.d. | n.d. | n.d. | n.d. | n.d. | n.d. | n.d. | n.d. | 0.34 | n.d. |
| | | | | | | | | | | | | | | border | |
| before | A4 218 | f | 79 | 1^{st} | AE | pos | pos | cont | cont | MAG | 2 | MIAG | 1 | 0.97 pos | 71.1 pos |
| | | | | 2^{nd} | N | neg | neg | neg | neg | MICG atr | 0 | MICG | 0 | n.d. | 1.2 neg |
| 3 months | | | | | n.d. | n.d. | n.d. | n.d. | n.d. | n.d. | n.d. | n.d. | n.d. | lost | n.d. |
| 5 months | | | | | n.d. | n.d. | n.d. | n.d. | n.d. | n.d. | n.d. | n.d. | n.d. | 0.73 pos | n.d. |
| before | A4 221 | f | 50 | 1^{st} | DE | pos | pos | pos | pos | MIAG | 1 | MIAG | 2 | 0.95 pos | 40.1 pos |
| | | | | 2^{nd} | DE | neg | neg | neg | neg | MICG atr | 0 | MICG atr | 0 | n.d. | 0.4 neg |
| 3 months | | | | | n.d. | n.d. | n.d. | n.d. | n.d. | n.d. | n.d. | n.d. | n.d. | 0.7 pos | n.d. |
| 5 months | | | | | n.d. | n.d. | n.d. | n.d. | n.d. | n.d. | n.d. | n.d. | n.d. | 0.62 pos | n.d. |
| before | A4 101 | m | 60 | 1^{st} | GU | pos | pos | pos | pos | MIAG | 2 | MIAG | 1 | 1.01 pos | 14.3 pos |
| | | | | 2^{nd} | N | neg | neg | neg | neg | MICG atr | 0 | MICG atr | 0 | n.d. | 0 |
| | | | | | | | | | | | | IM | | | |
| 3 months | | | | | n.d. | n.d. | n.d. | n.d. | n.d. | IM | n.d. | n.d. | n.d. | 0.77 pos | n.d. |
| 5 months | | | | | n.d. | n.d. | n.d. | n.d. | n.d. | n.d. | n.d. | n.d. | n.d. | 0.93 pos | n.d. |
| | | | | | | | | | | n.d. | | | | | |
| before | A4 102 | m | 73 | 1^{st} | GU | pos | pos | pos | pos | MIAG | 3 | MIAG | 1 | 0.88 pos | 45.1 pos |
| | | | | 2^{nd} | DE | neg | neg | neg | neg | MICG atr | 0 | MICG | 0 | n.d. | 0.0 neg |
| 3 months | | | | | n.d. | n.d. | n.d. | n.d. | n.d. | n.d. | n.d. | n.d. | n.d. | 0.80 pos | n.d. |
| 5 months | | | | | n.d. | n.d. | n.d. | n.d. | n.d. | n.d. | n.d. | n.d. | n.d. | 0.85 pos | n.d. |

N = normal;
AG = antral gastritis;
AE = antral erosions;
ED = erosive duodenitis;
SAG = severe gastritis;
DU = duodenal ulcer;
GU = gastric ulcer
atr = atrophy;
IM = intestinal metaplasia;
D = dysplasia;
MIAG = mild active gastritis;
MICG = mild inactive gastritis
m = male;
f = female;
A = antrum;
C = corpus;
n.d. = not done;
u.e. = under evaluation

TABLE 2
List of non-infected patients

| | | | | | Clo | | Colt | | | | | | | |
|---|---|---|---|---|---|---|---|---|---|---|---|---|---|---|
| [t]therapy | Patient | Sex | Age | ED | A | Clo C | A | Colt C | Histology A | Glemsa A | Histology C | Glemsa C | IgG | ¹³C UBT |
| before | A 449 | f | 56 | AG | neg | neg | neg | neg | N | 0 | N | 0 | 0.14 neg | 0.31 neg |
| before | A 459 | m | 41 | DE | neg | neg | neg | neg | N | 0 | N | 0 | 0.17 neg | neg |
| before | A 454 | m | 33 | AG | neg | neg | neg | neg | N | 0 | N | 0 | 0.17 neg | 0.05 neg |
| before | A 512 | f | 22 | N | neg | neg | neg | neg | N | 0 | N | 0 | 0.10 neg | 0.24 neg |
| before | A 569 | m | 52 | AG | neg | neg | neg | neg | MAG atr | 0 | N | 0 | 0.07 neg | 0.94 neg |
| before | A4 219 | m | 24 | AG | neg | neg | neg | neg | MICG | 0 | N | 0 | 0.10 neg | neg |
| before | A4 222 | f | 23 | AG | neg | neg | neg | neg | N | 0 | N | 0 | 0.10 neg | 1.5 neg |
| before | A4 227 | f | 58 | AG | neg | neg | neg | neg | MICG | 0 | N | 0 | 0.16 neg | 1.4 neg |
| before | A4 229 | f | 62 | AG | neg | neg | neg | neg | MICG atr | 0 | MICG | 0 | 0.24 neg | neg |
| before | A4 223 | m | 55 | AE | neg | neg | neg | neg | MICG | 0 | N | 0 | 0.27 neg | neg |

N = normal;
AG = antral gastritis;
AE = antral erosions;
ED = erosive duodenitis;
SAG = severe gastritis;
DU = duodenal ulcer;
GU = gastric ulcer
atr = atrophy;
IM = intestinal metaplasia;
D = dysplasia;
MIAG = mild active gastritis;
MICG = mild inactive gastritis
m = male;
f = female;
A = antrum;
C = corpus;
n.d. = not done;
u.e. = under evaluation

SDS-Polyacrylamide Gel Electrophoresis (SDS-PAGE) and Western-Blot Analysis:

The protein content of the bacterial lysates was determined by the method according to Lowry (Lowry et al., 1951) with bovine serum albumin as a standard. The lysates were dried in a speedVac concentrator. Afterwards the pellets were suspended in gel sample buffer (4% SDS, 12% glycerin, 4% β-mercaptoethanol, 0.01% Serva Blue G250 in 50 mM Tris/HCl pH 6.8) and boiled for 10 min. H. pylori and C. jejuni lysates were separated on 1.0 or 1.5 mm 7.5-16.5% SDS-tricine gradient gels (Schaegger and v. Jagow, 1987). Therefore a 7.5% and a 16.5% acrylamide solution for the separating gel and a 4% acrylamide solution for the stacking gel were prepared according to the scheme presented in table 3. Using a gradient-mixer the gel was poured slowly, but with continuous flow between the glass plates of the gel-sandwich. The separating gel was overlaid with deonized H₂O and allowed to polymerize for 1 hr. Afterwards the water was removed, APS and TEMED was added to the stacking gel solution that was poured on top of the separating gel. A comb with the appropriate number of sample pockets was inserted and removed after polymerisation for 1 hr. The gel-sandwich was placed in an electrophoresis chamber half filled with bottom running buffer (0.2 M Tris-HCI, pH 8.9). The upper compartment was filled with top running buffer (0.1 M Tris base, 0.1 M Tricine, pH 8.25). The protein samples (preparation see above) were loaded into the pockets and electrophoresis was performed with 15 mA constant current over night. Low or broad range prestained molecular mass standards (BioRad, Hercules, Calif., USA) were separated in parallel on each gel. The protein pattern after electrophoresis was determined by Silver staining (Heukeshoven, 1985) or Coomassie blue staining. For calculation of molecular weights RFPL scan software (Version 2.01, Scanalytics) was used.

TABLE 3
Composition of the Tricine gradient gel:

| | Stacking | | | | |
|---|---|---|---|---|---|
| | gel | | | | |
| | 4% | Separating gel | | | |
| | 1.0 mm/ | 7.5% | | 16.5% | |
| | 1.5 mm | 1.0 mm | 1.5 mm | 1.0 mm | 1.5 mm |
| Acrylamide solution | 1 ml | 3.05 ml | 5.00 ml | | |
| I | | | | | |
| Acrylamide solution | | | | 6.66 ml | 10.0 ml |
| II | | | | | |
| Gel buffer | 3 ml | 6.66 ml | 10.00 ml | 6.66 ml | 10.0 ml |
| Glycerol | | | | 1.33 g | 2 g |
| H₂O | 8.4 ml | 10.3 ml | 15.0 ml | 5.3 ml | 10.0 ml |
| 10% APS in H₂O | 100 μl | 40 μl | 50 μl | 40 μl | 50 μl |
| TEMED | 10 μl | 3.75 μl | 5 μl | 3.75 μl | 5 μl |

Acrylamide solution I: 48% (w/v) acrylamide, 1.5 (w/v) bisacrylamide (BioRad, Hercules, CA, USA) in H2O
Acrylamide solution II: 46.5% (w/v) acrylamide, 3.0% (w/v) NN′-methylene-bis-acrylamide (BioRad, Hercules, CA, USA) in H2O
Gel buffer: 3M Tris-HCl, pH 8.45: 0.3% SDS
APS = ammoniumpersulfate,
TEMED = N, N, N′, N′, tetramethylendiamin,
SDS = sodiumdodecylsulphate,
* freshly prepared
** added before the gel is poured

Quantitative Western-Blot Analysis

The reactivities of the antibodies in the sera with the antigens of interest were evaluated by imaging the autoradiographs with a Radioanalytic Imaging System (Ambis QuantProbe™ Software, version 4.31) and using RFPLScan® (version 2.01, Scanalytics) for determining the integrated optical density (IOD) of the protein bands of interest using a Gaussian calculation method provided with the program. The IOD of the particular protein band before treatment was set at 100%. The change of the reactivity, reflecting the titer of specific antibodies in the sera, during the post treatment period was calculated compared to the 100% level.

### Protein Sequencing:

Identification of Antigens

Isoelectric Focusing (IEF) and 2D gel Electrophoresis

Isoelectic focusing (IEF) was performed using the Multiphor II system (Amersham Pharmacia, NJ) and 11 cm Immobiline DryStrip gels with a pH range of 3-11. The DryStrips were re-hydrated overnight in 8 M urea, 0.5% NP40, 1% DTT at room temperature. H. pylori lysates containing up to 250 μg protein and prepared as described above were resuspended in 9 M urea, 2.0% NP40, 2.0% DTT, 0.8% IPG buffer pH 3-11 (ampholyte-containing buffer concentrate, Amersham Pharmacia) and loaded onto the re-hydrated gel strip. IEF was performed for 22 hr with 300 V and 0.04 mA/strip (4 hr) and 1900 V and 0.04 mA/strip (18 hr). The gel strips were then incubated for 2D SDS-PAGE first in 6 M urea in 4% SDS, 12% glycerol, 50 mM Tris/HCL, pH 6.5, 0.01% Serva Blue G250, 2% DTT (30 min) and then in 6 M urea in 4% SDS, 12% glycerol, 50 mM Tris/HCL, ph 6.5, 0.01% Serva Blue G250, 8% iodoacetamide (15 min), SDS-PAGE was performed in 1.0 or 1.5 mm 7.5-16.5% tricine-SDS gradient gels after embedding the IEF gel strips in 1% low-melting agarose, 1M Tris/HCL, pH 8.45, 0.1% SDS onto the stacking gel. Subsequently, the gels were Coomassie Blue stained or electro-transferred onto nitrocellulose membranes for Western blot analysis.

Identification of Proteins by Mass Spectrometry

For mass-spectrometry, H. pylori lysates underwent 2D gel electrophoresis and subsequent separation on a tricine gradient gel followed by Coomassie blue staining and Western blotting. The protein spots at the Mwt of interest were excised from the acrylamide gel and analyzed using mass spectrometry after in situ digestion (performed at the Mass Spectrometry Core Facility, Division of Immunology, Beckman Research Institute City of Hope, Duarte, Calif.). The peptide fragments were used for a homology search based on Mwt and iso-electric point in the database to identify the proteins of H. pylori containing the peptides. Four immuno-reactive spots were found in the low Mwt range; HP1 at 32 kDa with a pI of ˜7.5, HP2 at 30 kDa with a pI of ˜6.1; HP3 at 22 kDa with pI of ˜8.9; and HP4 at 14 kDa with a pI of ˜5.9. These proteins were excised from the Coomassie stained 2D gel and analyzed by mass-spectrometry.

Expression and Purification of 6×His Tagged Recombinant Proteins

The identified antigens were expressed and purified using the QIAexpressionist™ system (Qiagen, Valencia, Calif.). The coding sequences of the 8 proteins were cloned either as ATG constructs into pQE60 providing a C-terminal 6×His tag or into pQE30 providing an N-terminal 6×His tag in case of HP0596 and ATPF₀b′ and transformed into E. coli M15. Where applicable, it was necessary to remove the signal peptides for cloning and expression (see Table 2). Expression and purification of the recombinant proteins was performed under denaturing conditions with Ni-NTA agarose. The antigenic reactivity of the recombinant proteins was determined by SDS-PAGE on 10% tricine-SDS mini-gels and Western blot analysis using the positive or control sera.

Two proteins were found to be present at each of the four spots yielding a total of eight proteins for further analysis by immune reactivity. Two were outer membrane proteins; Omp 18, identified by 5 distinct peptides with 29.1% sequence coverage and HpaA, identified by 10 distinct peptides covering 42.7% of the sequence.

Two of the proteins were associated with the inner membrane: ATP-F0b′ identified by 4 distinct peptides with 29.2% sequence coverage and HP0596 identified by 6 peptides giving 40.1% sequence coverage. Four were cytoplasmic proteins: CoA-trans identified by 7 peptides (48.3% sequence coverage), TagD by 3 peptides (32.3% sequence coverage), EF-P identified by 5 peptides with 36.4% of the sequence covered and RPL7/L12 identified by 7 peptides covering 70% of the protein sequence.

Immune Response Towards the Recombinant Proteins

To define which of these eight proteins was recognized by human sera, all eight sequences were expressed as recombinant proteins with a 6×H is epitope (QiaExpressionist™ system, Qiagen, Valencia, Calif., USA). It was found that the presence of a signal peptide often hindered expression and hence was deleted for recombinant expression and in one case, the 6×His tag was placed on the N terminus. The results of recombinant expression are summarized in Table 4. Subsequently, the recombinant proteins were analyzed by Western blotting using an antibody against the 6×His tag (Penta-His™ antibody, Qiagen) to confirm expression. Results of immunoblotting the recombinant proteins with patient sera are shown in Table 5.

TABLE 4
Identification of the H. pylori antigens by
mass spectrometry, recombinant expression and purification

| | | | | | | C- or N- |
|---|---|---|---|---|---|---|
| | | Relative | | | | term 6xHis |
| | | Mwt | | | recombinant | Tag |
| Spot | Antigen | kDa | pI | TIGR ID | expression | +/− SP |
| HP1(a) | Neuraminyl-lactose- | 32 | 7.5 | HP0797 | +++ | C- |
| | binding Hemagglutinin | | | | | term − SP |
| | precursor HpaA | | | | | |
| Hp1(b) | 3-Oxoacid COA | 32 | 7.5 | HP0691 | +++ | C-term |
| | transferase subunit A | | | | | no SP |
| | CoA-trans | | | | | |
| HP2(a) | Elongations factor P | 30 | 6.1 | HP0177 | +++ | C-term |
| | EF-P | | | | | no SP |
| HP2(b) | Peptidogylcan | 30 | 6.1 | HP1125 | +++ | C- |
| | associated lipoprotein | | | | | term − SP |
| | precursor Omp18 | | | | | |
| HP3(a) | Adhesin-thiol | 22 | 8.9 | HP0390 | + | C-term |
| | peroxidase TagD | | | | | no SP |
| HP3(b) | Hypothetical protein | 22 | 8.9 | HP0596 | +++ | N- |
| | HP0596 | | | | | term − SP |
| HP4(a) | Ribosomal protein | 14 | 5.9 | HP1199 | +++ | C-term |
| | L7/L12 RPL7/L12 | | | | | no SP |
| HP4(b) | ATP Synthase F₀ | 14 | 5.9 | HP1137 | ++ | N- |
| | subunit b′ ATP-F₀b′ | | | | | term − SP |

As is known in the art TIGR ID refers to identification from a widely available known genomic data base.

TABLE 5
Antigenic profile of the low Mwt recombinant
antigens with H. pylori positive and negative sera.

| | | | | | | | | | | | | | | | |
|---|---|---|---|---|---|---|---|---|---|---|---|---|---|---|---|
| 2D Spot | Antigen | P1 | P2 | P3 | P4 | P5 | P6 | P7 | P9 | P10 | N1 | N2 | N3 | N4 | N5 |

| | | | | | | | | | | | | | | | |
|---|---|---|---|---|---|---|---|---|---|---|---|---|---|---|---|
| HP1(a) | HPaA | − | − | + | + | + | + | (+) | + | + | − | − | − | − | − |
| HP1(b) | Co-A- | − | − | − | − | − | − | − | − | − | − | − | − | − | − |
| | trans | | | | | | | | | | | | | | |
| HP2(a) | EF-P | − | − | − | − | − | − | − | − | − | − | − | − | − | − |
| HP2(b) | Omp18 | + | + | + | + | + | + | + | + | + | − | − | − | − | − |
| HP3(a) | HP0596 | − | − | + | + | + | + | + | + | + | − | − | − | − | − |
| HP3(b) | TagD | − | − | − | − | − | − | − | − | − | − | − | − | − | − |
| HP4(a) | RPL7/L12 | + | + | − | + | − | + | + | + | + | − | − | − | − | + |
| HP4(b) | ATP-F₀b′ | − | (+) | − | − | − | − | − | − | − | − | − | − | − | − |

### EXAMPLES

SDS-PAGE and Immunoblot Analysis:

The separation of whole cell lysates of three H. pylori strains and one C. jejuni strain on tricine gradient gels was performed to show the protein patterns by Coomassie blue or silverstaining of the gels.

The following immunoblot analysis probing the membranes with the ten human sera from non-infected individuals showed that there were only a few proteins of H. pylori reacting with these sera. All the proteins reacting with the negative sera were mainly found in the higher molecular weight range and are probably proteins being homologous to proteins from other bacterial species and therefore causing cross reactivities with antibodies generated during infection with H. pylori. A similar result was seen with C. jejuni proteins supporting that these reactions are supposed to be considered as non specific.

Probing the membranes with the nine human sera from H. pylori infected patients obtained before eradication treatment showed again some cross reactivities with C. jejuni proteins being either in the high molecular weight range or definitely different from proteins recognized in the tested H. pylori strains by these sera.

Using specific sera against both subunits of urease from H. pylori, urease A and urease B, and one of the heat shock proteins, HspB, showed that the antigens according the invention are different from these H. pylori proteins.

All of the four antigens that are subject of this invention were clearly recognized by eight of the investigated sera from H. pylori infected patients in all three tested H. pylori strains (Hp504, Hp08, Hp02). One of the sera (HS#0 12, table 1) did not react with one of the antigens (HP4).

Quantification of the Reactivities with the H. pylori Antigens:

The four immuno-reactive antigens described in this invention were visualized by using the ECL™ detection system. The reactivities of the four antigens of interest were evaluated by imaging the autoradiographs with a Radioanalytic Imaging System and using specialized software (RFPLScan® version 2.01) for determining the integrated optical density (IOD) of each single antigen at the different time points (before treatment, 3 months and 5 months after treatment) from the three different H. pylori strains. The IOD of each antigen before eradication therapy was set as 100% on each immunoblot that was evaluated. The changes in the reactivities of the sera with these antigens could also be looked at showing the changes in titers of specific serum IgG antibodies against HP1, HP2, HP3 and HP4.

The following figures show the serial changes in titers of serum IgG expressed in % of integrated optical density that is left 3 months and 5 months after eradication treatment in comparison to the amount before treatment. The nine sera from with H. pylori infected and treated patients were tested on whole cell lysate separations of H. pylori strain ATCC#43504 (Hp504), Hp08 and Hp02 (clinical isolates). The results are shown for each tested bacterial strain separately to demonstrate that the accuracy of the test is independent of the source of the antigen.

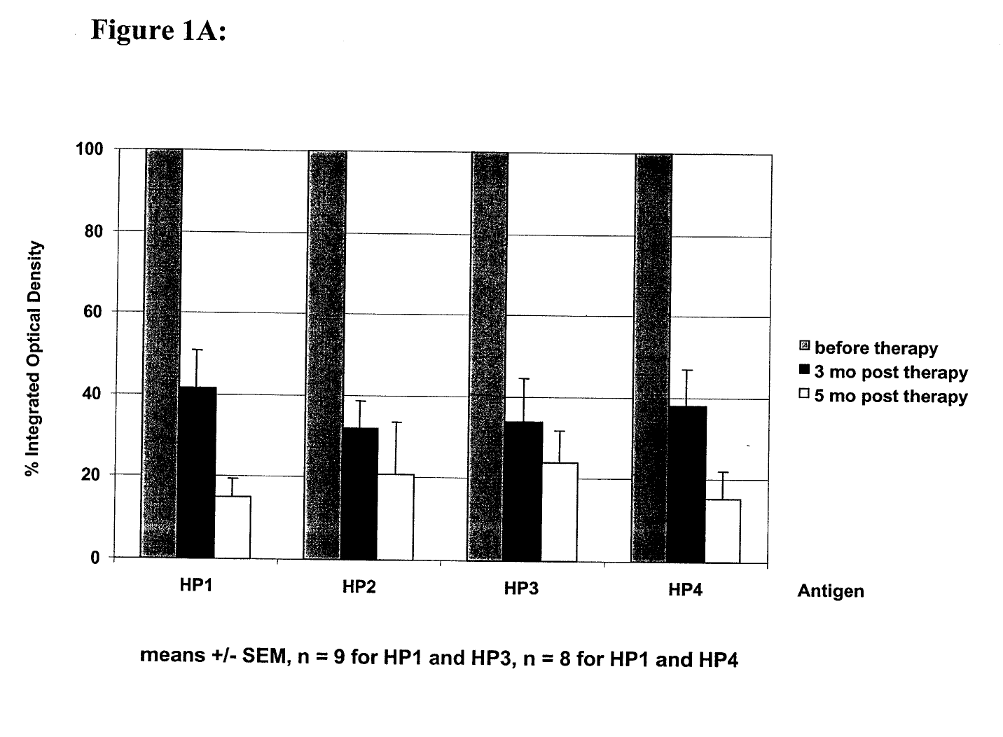

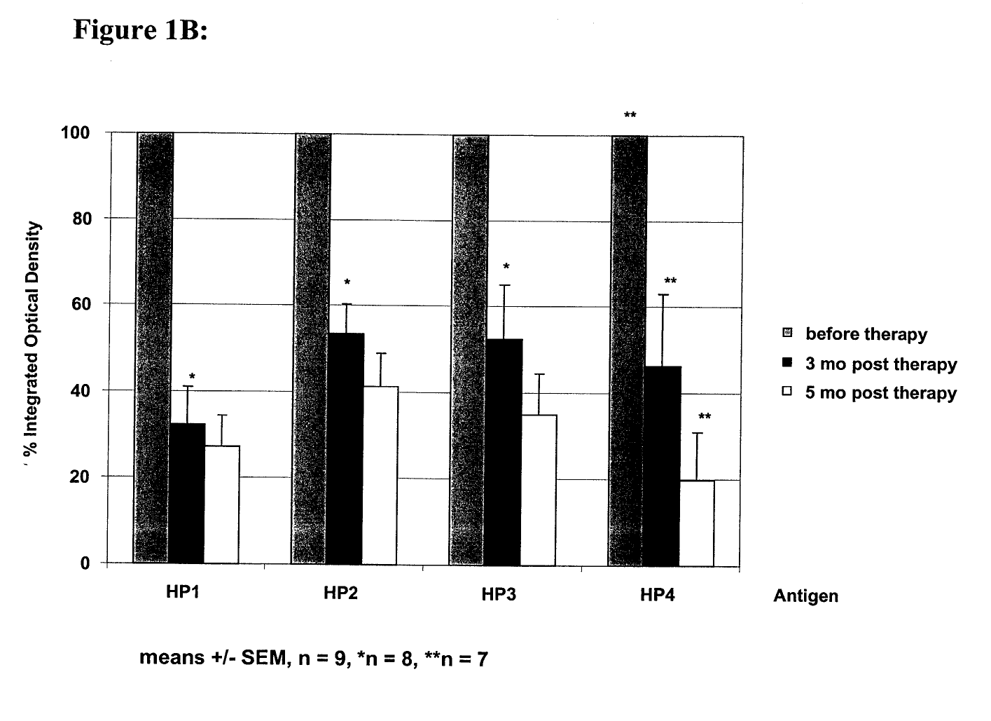

FIGS. 1A and B show the data for the sera being tested on Hp504 antigen preparations in two independent experiments. Differences between the two data sets obtained with antigens from strain Hp504 show that the results may depend to some extent on the antigen preparation itself and/or the performance of the Western-blot analysis. However, on the basis of this disclosure a person of ordinary skill in the art can readily standardize the parameters of a practical test kit without undue experimentation. The data on which the charts of FIGS. 1A and 1B are based are disclosed in Tables 6 and 7, respectively.

TABLE 6

| t/therapy | % IOD HP1 | % IOD HP2 | % IODHP3 | % IODHP4 |
|---|---|---|---|---|
| before | 100 | 100 | 100 | 100 |
| 3 months post | 41.61 +/− 9.25 | 32.08 +/− 6.48 | 33.88 +/− 10.59 | 37.86 +/− 8.98 |
| 5 months post | 15.15 +/− 4.20 | 20.71 +/− 12.72 | 23.81 +/− 7.83 | 15.37 +/− 6.68 |

TABLE 7

| t/ | % IOD | % IOD | | |
|---|---|---|---|---|
| therapy | HP1 | HP2 | % IODHP3 | % IODHP4 |
| before | 100 | 100 | 100 | 100 |
| 3 | 32.15 +/− | 53.58 +/− | 52.37 +/− 12.72 | 46.17 +/− 16.87 |
| months | 8.85 | 6.81 | | |
| post | | | | |
| 5 | 27.2 +/− | 41.28 +/− | 34.77 +/− 9.61 | 19.77 +/− 11.11 |
| months | 7.05 | 7.64 | | |
| post | | | | |

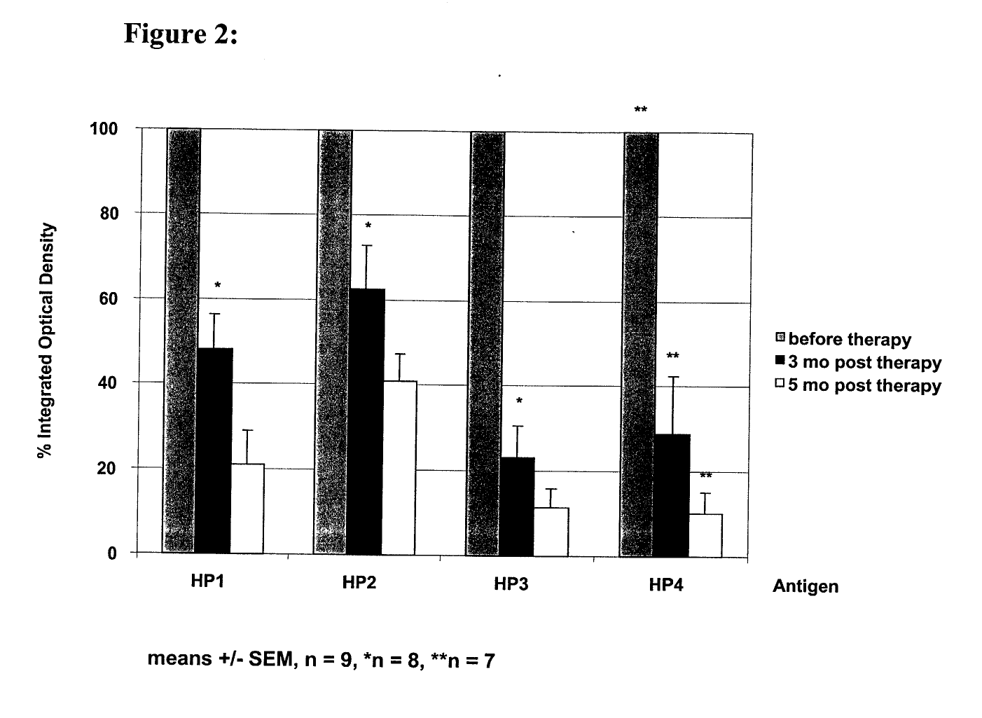

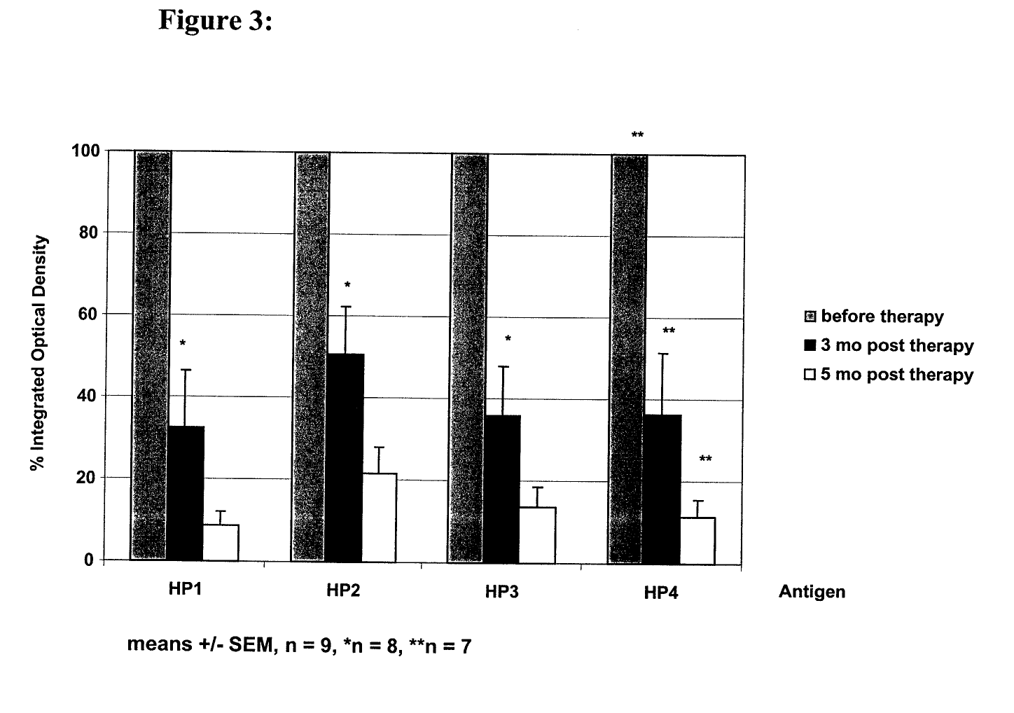

FIGS. 2 and 3 show the results for the sera being tested on Hp08 and Hp02 antigen preparations respectively. The data on which the charts of FIGS. 2 and 3 are based are disclosed in Tables 8 and 9, respectively.

TABLE 8

| t/therapy | % IOD HP1 | % IOD HP2 | % IODHP3 | % IODHP4 |
|---|---|---|---|---|
| before | 100 | 100 | 100 | 100 |
| 3 months post | 48.11 +/− 8.15 | 62.53 +/− 10.32 | 23.05 +/− 7.53 | 28.60 +/− 13.85 |
| 5 months post | 21.06 +/− 8.01 | 40.77 +/− 4.45 | 11.36 +/− 4.45 | 9.96 +/− 4.99 |

TABLE 9

| t/therapy | % IOD HP1 | % IOD HP2 | % IODHP3 | % IODHP4 |
|---|---|---|---|---|
| before | 100 | 100 | 100 | 100 |
| 3 months post | 32.30 +/− 13.97 | 50.71 +/− 11.48 | 35.86 +/− 11.88 | 36.29 +/− 14.99 |
| 5 months post | 8.70 +/− 3.2 | 21.56 +/− 6.44 | 13.51 +/− 5.02 | 11.39 +/− 4.27 |

In all cases there was a significant decrease detected in the reactivities of the nine sera with the five antigens 3 months after therapy that increased further 5 months after treatment. The detected decrease in titers of H. pylori specific antibodies shows eradication of the infection what is supported by the results of the other tests that were performed on the patients (Table 8).

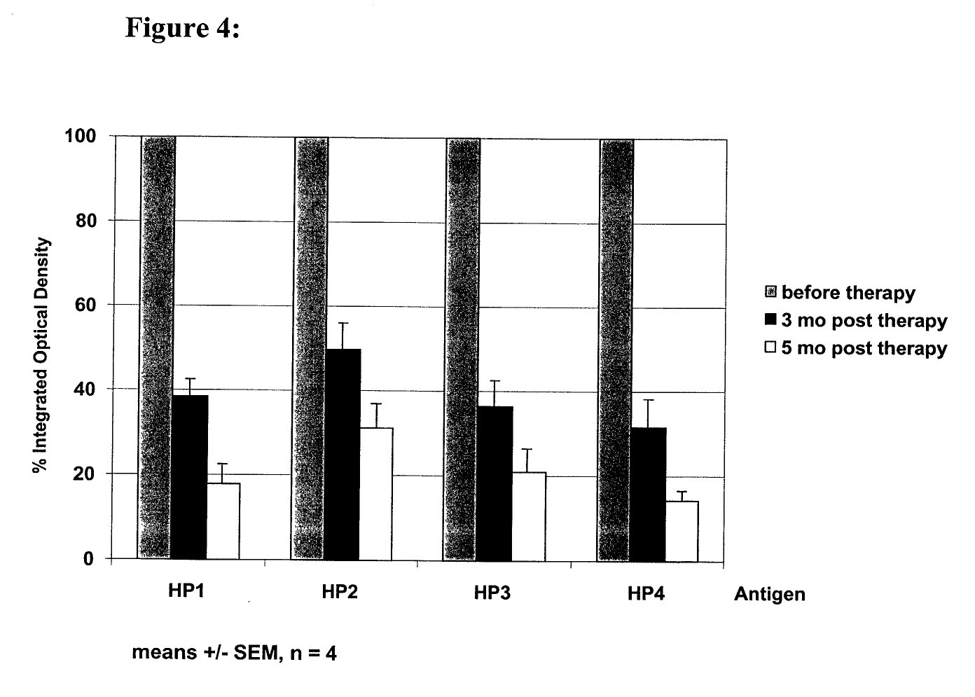

FIG. 4 summarizes the data of the previous experiments and shows the average titers of specific antibodies against HP1, HP2, HP3, HP4 from all four different H. pylori strains in the patient's sera. As shown here the average titer of anti-HP1 antibodies at 3 months after eradication treatment decreased to 38.5% (=61.5% reduction) and to 18.03% (=81.97% reduction) at 5 months after end of treatment respectively. The average titer of anti-HP2 antibodies found at 3 months is down to 49.73% (=50.27% reduction) and to 31.08% (=68.92% reduction) at 5 months after therapy respectively. For anti-HP3 antibodies there is a decrease in the average titer to 36.29% (=63.71% reduction) at 3 months and a further decrease to 28.87% (=79.13% reduction). Finally the average titer of anti-HP4 antibodies at 3 months is down to 31.61% (=68.39% reduction) and to 14.12% (=85.88% reduction) at 5 months after therapy.

Accuracy of a Combination of HP1, HP2, HP3 and HP4 in a Test Set:

A combination of the described four antigens from H. pylori on a Western-blot test strip applying the correct cut-off setting for each of the antigens provides a sensitive test for the diagnosis of an infection with H. pylori, for monitoring the early response to eradication therapy and for determining the eradication of the infection. It is preferred to provide a test strip that contains all of the investigated antigens because the study showed that one or the other of the antigens is recognized differently by the different sera. Providing the combination and not a mixture of HP1, HP2, HP3 and HP4 on a strip also decreases drop-outs if a serum fails to react with one of the antigens. Table 10 shows the cut-off setting for each of the antigens. It is believed that providing the four antigenic proteins HP1, HP2, HP3 and HP4, or least three of these four proteins, in a test plate or test membrane at four (or three) different locations, namely as a combination rather than a mixture, is a unique aspect of the present invention. This feature renders highly reliable and accurate the diagnosis of infection by H. pylori as well the process of quantitatively monitoring the eradication of these bacteria by drug therapy.

TABLE 10
Cut-off setting for H. pylori antigens used in test kit.

| | cut-off at | cut-off at |
|---|---|---|
| Antigen | 3 months post therapy | 5 months post therapy |
| HP1 | 58% titer decrease | 78% titer decrease |
| HP2 | 44% titer decrease | 63% titer decrease |
| HP3 | 58% titer decrease | 74% titer decrease |
| HP4 | 62% titer decrease | 84% titer decrease |

What is claimed is:

1. A composition comprising at least three proteins, wherein the proteins is selected from the group consisting of HP1, HP2, HP3 and HP4, each of said proteins comprising regions which act as antigens specific to Helicobacter pylori, HP1 having of molecular weight of 32 kd, HP2 having of molecular weight of 30 kd, HP3 having of molecular weight of 23 kd, and HP4 having of molecular weight of 15 kd, each of said proteins being derived from Helicobacter pylori bacteria.

2. A composition according to claim 1 wherein:
HP1 has the sequence of
1 mkkgslaivl gsllasgafy taladgmpak qqhnntgesv elhfhypikg kqepknshlv
61 vliepkiein kvipesyqke fekslflqls sflerkgysv sqfkdaseip qdikekallv
121 lrmdgnvail ediveesdal seekvidmss gylnlnfvep ksediihsfg idvskikavi
181 ervelrrtns ggfvpktfvh riketdhdqa irkimnqayh kvmvhitkel skkhmehyek
241 vssemkkrk
HP2 has the sequence of
1 mkrssvfsfl vafllvagcs hkmdnktvag dvsaktvqta pvttepapek eepkqepapv
61 veekpavesg tiiasiyfdf dkyeikesdq etldeivqka kenhmqvlle gntdefgsse
121 ynqalgvkrt lsvknalvik gvekdmikti sfgetkpkca qktrecyken rrvdvklmk
HP3 has the sequence of
1 mleksflksk qlflcglgvl mlqactcpnt sqrnsflqdv pywmlqnrse yitqgvdssh
61 ivdgkkteei ekiatkrati rvaqnivhkl keaylsktnr ikqkitnemf iqmtqpiyds
121 lmnvdrlgiy inpnneevfa lvrargfdkd alseglhkms ldnqavsilv akveeifkds
181 vnygdvkvpi am
HP4 has the sequence of
1 mnisvnpylm avvfvvfvll lwamnvwvyr pllafmdnrq aeikdslaki ktdnaqsvei
61 ghqieallke aaekrreiia eaiqkatesy davikqkene lnqefeafak qlqnekqalk
121 eqlqaqmpvf edelnkrvam glgs.

3. A composition according to claim 2, wherein each of the proteins of the composition are in purified state.

4. A composition according to claim 2, wherein all four of said proteins are present.

5. A composition according to claim 2, which is a combination and not a mixture of said proteins.

6. A composition according to claim 2 wherein the HP1, HP2 and HP4 proteins are present.

7. A composition according to claim 2, wherein said proteins are present attached to a suitable solid phase.

8. A composition according to claim 7, wherein the solid phase is a microtiter plate.

9. A composition according to claim 8, wherein the proteins are present attached to membranes.

10. A composition according to claim 9 wherein the membranes are nitrocellulose or PVDF membranes.

11. A composition according to claim 7, wherein a combination and not a mixture of proteins is provided on a test strip.

12. A method for the preparation of a composition according to claim 10 by preparing a lysate of whole bacterial cell preparations of Helicobacter pylori, subjecting the lysate to gel separation and transferring the proteins to the membranes.

13. A method according to claim 12 wherein the Helicobacter pylori is Helicobacter pylori strain ATCC#43504.

14. A method according to claim 12 wherein the gel separation is carried out in 7.5-16.5% SDS-tricine gradient gels.

15. A method for detecting the presence of antibodies resulting from Helicobacter pylori infection in a biological sample, the method comprising
contacting the sample with a composition according to claim 2,
permitting the sample and said composition to form an antigen-antibody complex with respect to any antibody contained in the sample which specific to the antigens included in the proteins of the composition;
detecting the presence of any formed antigen-antibody complex denoting the presence of Helicobacter pylori infection.

16. A method according to claim 15 wherein in the step of detecting an enzyme-conjugated anti-Human IgG antibody is used for detection of the antigen-antibody complex.

17. A method according to claim 16 wherein the anti-Human IgG antibody is conjugated to horseradish peroxidase.

18. A method according to claim 15 wherein in the step of detecting gold labeled antibody is used for detection of the antigen-antibody complex.

19. A method according to claim 15 wherein the biological sample is human serum.

20. A kit for determining the presence of antibodies formed in response to Helicobacter pylori infection in a biological sample, the kit comprising a composition according to claim 2.

21. A kit according to claim 20 wherein a combination and not a mixture of the proteins is provided on a test strip.

22. A kit according to claim 20 additionally comprising a positive control, and an enzyme-conjugated antiHuman IgG antibody.

23. A kit according to claim 22 additionally comprising a suitable enzyme substrate and buffer solution.

24. A kit according to claim 20 comprising a test strip, wherein the composition is attached to a nitrocellulose membrane and a gold labeled antibody is used for detection.

25. In a method for determination the eradication of Helicobacter pylori the improvement consisting in the detection of the presence or absence of antibodies resulting from Helicobacter pylori infection by a method according to claim 15, before, during and after eradication treatment.

26. A method of using a combination of at least 3 proteins from Helicobacter pylori for detecting the presence or absence of antibodies resulting from Helicobacter pylori infection wherein the proteins are selected from the group consisting of HP1, HP2, HP3 and HP4, each of said proteins comprising regions which act as antigens specific to Helicobacter pylori, HP1 having of molecular weight of 32 kd, HP2 having of molecular weight of 30 kd, HP3 having of molecular weight of 23 kd, and HP4 having of molecular weight of 15 kd, each of said proteins being derived from Helicobacter pylori bacteria.

27. A method according to claim 26 wherein
HP1 has the sequence of
1 mkkgslaivl gsllasgafy taladgmpak qqhnntgesv elhfhypikg kqepknshlv
61 vliepkiein kvipesyqke fekslflqls sflerkgysv sqfkdaseip qdikekallv
121 lrmdgnvail ediveesdal seekvidmss gylnlnfvep ksediihsfg idvskikavi
181 ervelrrtns ggfvpktfvh riketdhdqa irkimnqayh kvmvhitkel skkhmehyek
241 vssemkkrk
HP2 has the sequence of
1 mkrssvfsfl vafllvagcs hkmdnktvag dvsaktvqta pvttepapek eepkqepapv
61 veekpavesg tiiasiyfdf dkyeikesdq etldeivqka kenhmqvlle gntdefgsse
121 ynqalgvkrt lsvknalvik gvekdmikti sfgetkpkca qktrecyken rrvdvklmk
HP3 has the sequence of
1 mleksflksk qlflcglgvl mlqactcpnt sqrnsflqdv pywmlqnrse yitqgvdssh
61 ivdgkkteei ekiatkrati rvaqnivhkl keaylsktnr ikqkitnemf iqmtqpiyds
121 lmnvdrlgiy inpnneevfa lvrargfdkd alseglhkms ldnqavsilv akveeifkds
181 vnygdvkvpi am
HP4 has the sequence of
1 mnisvnpylm avvfvvfvll lwamnvwvyr pllafmdnrq aeikdslaki ktdnaqsvei
61 ghqieallke aaekrreiia eaiqkatesy davikqkene lnqefeafak qlqnekqalk
121 eqlqaqmpvf edelnkrvam glgs.


SEQUENCE LISTING (4 sequences)

SEQ ID NO: 1 - protein, 249 aa, Helicobacter pylori
MKKGSLAIVLGSLLASGAFYTALADGMPAKQQHNNTGESVELHFHYPIKGKQEPKNSHLVVLIEPKIEINKVIPESYQKEFEKSLFLQLSSFLERKGYSVSQFKDASEIPQDIKEKALLVLRMDGNVAILEDIVEESDALSEEKVIDMSSGYLNLNFVEPKSEDIIHSFGIDVSKIKAVIERVELRRTNSGGFVPKTFVHRIKETDHDQAIRKIMNQAYHKVMVHITKELSKKHMEHYEKVSSEMKKRK

SEQ ID NO: 2 - protein, 179 aa, Helicobacter pylori
MKRSSVFSFLVAFLLVAGCSHKMDNKTVAGDVSAKTVQTAPVTTEPAPEKEEPKQEPAPVVEEKPAVESGTIIASIYFDFDKYEIKESDQETLDEIVQKAKENHMQVLLEGNTDEFGSSEYNQALGVKRTLSVKNALVIKGVEKDMIKTISFGETKPKCAQKTRECYKENRRVDVKLMK

SEQ ID NO: 3 - protein, 192 aa, Helicobacter pylori
MLEKSFLKSKQLFLCGLGVLMLQACTCPNTSQRNSFLQDVPYWMLQNRSEYITQGVDSSHIVDGKKTEEIEKIATKRATIRVAQNIVHKLKEAYLSKTNRIKQKITNEMFIQMTQPIYDSLMNVDRLGIYINPNNEEVFALVRARGFDKDALSEGLHKMSLDNQAVSILVAKVEEIFKDSVNYGDVKVPIAM

SEQ ID NO: 4 - protein, 144 aa, Helicobacter pylori
MNISVNPYLMAVVFVVFVLLLWAMNVWVYRPLLAFMDNRQAEIKDSLAKIKTDNAQSVEIGHQIEALLKEAAEKRREIIAEAIQKATESYDAVIKQKENELNQEFEAFAKQLQNEKQALKEQLQAQMPVFEDELNKRVAMGLGS